(19) United States
(12) Patent Application Publication
(10) Pub. No.: US 2026/0228885 A1
(43) Pub. Date: Aug. 6, 2026

(54) BIOMARKER IDENTIFIER AND ASSOCIATED METHOD

(71) Applicant: ARIZONA BOARD OF REGENTS ON BEHALF OF THE UNIVERSITY OF ARIZONA, Tucson, AZ (US)

(72) Inventors: Travis SAWYER, Tucson, AZ (US); Suzann DUAN, Tucson, AZ (US); Thomas KNAPP, Tucson, AZ (US)

(21) Appl. No.: 19/147,086

(22) PCT Filed: Feb. 6, 2024
(86) PCT No.: PCT/US2024/014684

§ 371 Date: Jul. 10, 2025

Related U.S. Application Data
(60) Provisional application No. 63/483,473, filed on Feb. 6, 2023.

(51) Int. Cl. G06T 7/00; G06V 10/26; G06V 10/77; G06V 20/69

(52) U.S. Cl. CPC G06T 7/0012; G06V 10/26; G06V 10/77; G06V 20/69; G06T 2207/30024

(57) ABSTRACT

A method for identify an imaging biomarker of an image of a tissue sample includes generating a spatial representation of an omics dataset of the tissue sample and coregistering the image and the spatial representation. The method also includes quantitatively extracting, as the imaging biomarker, imaging features from the coregistered image that are predictive of omic variation.

### CROSS-REFERENCE TO RELATED APPLICATIONS

This application claims the benefit of U.S. Provisional Application No. 63/483,473, filed on 6 Feb. 2023, the disclosure of which is incorporated herein by reference in its entirety.

### GOVERNMENT RIGHTS

This invention was made with government support under award number W81XWH-22-1-0211 granted by the Department of Defense and award numbers P30 CA023074 and T32 GM132008 awarded by the National Cancer Institute of the National Institutes of Health. The government has certain rights in the invention.

### BACKGROUND

Pancreatic neuroendocrine tumors (pNETs) are a group of neoplasms that have steadily increased in incidence over the last three decades, the majority being incidentally found small non-functional pNETs (sNF-pNETS). Present guidelines for the surgical treatment of PNETs recommend the resection of all pNETs greater than 2 cm in size. While a majority of pNETs are malignant, removal of indolent tumors exposes patients to unnecessary risks associated with surgery. Conservative approaches, such as ‘wait-and-see’ observation periods or smaller, lymph node preserving resections are thus used when treating sNF-pNETs. Conflicting findings on patient outcomes following both conservative and aggressive management of small NF-pNETs suggests that careful pre-operative assessment of tumor biology is necessary. Proper classification using the World Health Organization-American Joint Committee on Cancer (WHO-AJCC) pNET grading scale has been shown to accurately predict patient survival for both functional and NF-pNETs and is thus a useful aid in clinical decision-making. Accurate grading is challenged by the characteristically heterogeneous nature of pNETs, with evidence showing current practices of histological and cytological assessment resulting in inaccurate predictions of tumor grade. Incomplete sampling of pNET tumor masses during needle or surgical biopsy procedures introduce further risk of improper grading through the partial representation of tumor composition. Pathologists are thus challenged in the accurate grading of pNETs with the use of current methods such as measuring mitotic count and Ki-67 index.

### SUMMARY OF THE EMBODIMENTS

In a first aspect, a method for identify an imaging biomarker of an image of a tissue sample is disclosed. The method includes generating a spatial representation of an omics dataset of the tissue sample and coregistering the image and the spatial representation. The method also includes quantitatively extracting, as the imaging biomarker, imaging features from the coregistered image that are predictive of omic variation.

In a second aspect, a biomarker identifier is disclosed. The biomarker identifier includes a processor and a memory. The memory stores machine-readable instructions that, when executed by the processor, cause the processor to identify an imaging biomarker of an image of a tissue sample by executing the method of the first aspect.

### BRIEF DESCRIPTION OF THE FIGURES

FIG. 1 includes heatmaps showing comparisons of relative abundance of MPM fluorescence channel intensities.

FIG. 2 includes example images of PNET and DGAST acquired using MPM, both imaged with parameters set to acquire fluorescence predominantly from lipofuscin and lipofuscin-like fluorophores.

FIG. 3 illustrates Correlation heatmaps between genetic expression and MPM image channel intensities.

FIG. 4 is a fluorescence spectrum of primary fluorophores found in human tissue and their signal intensity over a broad band of excitation wavelengths.

FIG. 5 is a diagram of illustrating a method that includes spatial transcriptome sequencing, multiphoton imaging, and fluorescence lifetime imaging on serial sections of tissue, coregistering the datasets, and performing pixel-wise analysis between imaging and transcriptomic datasets.

FIG. 6 includes brightfield images of the four specimens used for biomarker identification. All four were collected from different patients diagnosed with pancreatic neuroendocrine tumors. Three samples had areas classified as tumor and three had areas classified as normal.

FIG. 7 shows Large morphologic similarities between serial sections were the primary target for placement of manual registration landmarks between the target H&E image (left) and label-free.

FIG. 8 illustrates registration of the label-free images to the Visium barcode coordinate space, which allows for the extraction of feature vectors containing both gene expression data and quantifiable properties of the images.

FIG. 9 shows Examples of multiphoton images tuned to different channels collected for each patient specimen. Every channel was collected for all patient specimens.

FIG. 10 shows high resolution examples of multiphoton images shown in FIG. 8 for each of our patient samples, illustrating significant tumor heterogeneity in our images (top row). In contrast, normal pancreatic tissue appears relatively consistent across different samples (bottom row).

FIG. 11 shows fluorescence lifetime imaging at 390 nm excitation for our patient specimens. The dotted lines for S2 and S3 show the tissue boundaries. Similar to the MPM images, we observe significant tumor heterogeneity, but consistent normal tissue characteristics.

FIG. 12 shows fluorescence lifetime imaging at 470 nm excitation for our patient specimens. The dotted lines for S2 and S3 show the tissue boundaries. Similar to the MPM images, we observe significant tumor heterogeneity, but consistent normal tissue characteristics.

FIG. 13 shows K-means clustering with k=7 performed for spatial transcriptomic data with all four patients pooled together. The clusters are superimposed on H&E images with each cluster positioned in the respective spatial barcode region.

FIG. 14 is a side-by-side comparison of multiphoton images, FLIm images at 390 nm excitation, and transcriptomic clusters superimposed on H&E images.

FIG. 15 includes (A) a visualization the expression of a single gene provides an equivalent to antibody staining. (B) An example of quantitative analysis to evaluate correlation between imaging markers and gene expression. Each point on the scatter plot represents a single barcode region. We see a relationship between our imaging markers and gene expression shown by two clear overlapping distributions.

FIG. 16 includes (A) K-means clustering of the gene expression data with k=3 of the barcode regions in patient S1. (B) Probability density distributions of the average of FAD/NADH channels for each barcode regions, grouped according to the three clusters. (C) Average values of each distribution show statistically significant differences using an independent t-test where *** denotes p<0.001.

FIG. 17 is a functional block diagram of a biomarker identifier, in an embodiment.

FIG. 18 is a flowchart of a method for identifying an imaging biomarker of an image of a tissue sample, in an embodiment.

### DETAILED DESCRIPTION OF THE EMBODIMENTS

The measurement of serum, genomic, and molecular markers have led to the determination of pNET subtypes that classify tumors with greater prognostic accuracy. Because these methods of biomarker extraction either do not sample the tumor directly or rely on bulk tissue analysis, they do not provide a full picture of local differences in cell composition within a heterogeneous tumor mass. With the introduction of mixed tumor types in the 2017 WHO update on the classification of pNETs, it seems necessary to achieve cell, or near-cell-specific sensitivity of tumor classification to achieve truly personalized treatment of this disease. Optical imaging provides several avenues for collecting whole-tissue information at potentially subcellular resolutions. Autofluorescent imaging acquires data that is a direct reflection of various cell states that effect the localization and abundance of endogenous fluorophores.

Embodiments disclosed herein use autofluorescent imaging features to model changes in tissue genetic expression and transcription.

### 1.1 Embodiments of Methods

Method 1: A method includes determining NF-pNET subtype classification, and possible existence of multiple subtypes within a single sample, through spatially resolved miRNA sequencing. The method may include separation of tumor regions within NF-pNET samples using laser-capture microdissection. This separation step allows for a step of determining of distinct NF-pNET subtypes based on their molecular information.

Method 2: A method includes determining and comparing genetic pathway variations and cell-type clusters in NF-pNET samples using spatial transcriptomics and their relationship to common prognostic guidelines and patient outcome. When distinct cell types may exist within the heterogeneous NF-pNETs, the method may include clustering the cell types in 2-D space based on their transcriptomic properties. The method may also include determining correlations between variations in cluster types and tumor metastatic risk, patient outcomes and prognosis using Ki-67/mitotic count.

Method 3: A method includes measuring and quantifying the autofluorescence spectrum of NF-pNET samples and develop a model of NF-pNET autofluorescence and genetic expression. Endogenous fluorophores and cell physiology are connected. The method may include deriving features from high-dimensional autofluorescent imaging and pairing said features with spatially resolved transcriptomics, and from said features and paired features, creating machine-learning models. The method may include using these models to describe changes in tissue fluorescence based on pathway variations, such as changes in cell metabolism, senescence, extracellular matrix composition, etc.

Method 4. A method for generating-omics data using optical imaging is disclosed. The method includes conducting spatial-omics on tissue samples along with label-free optical imaging, and coregistering the two datasets. The method also includes at least one of the following steps, which are based the coregistered data sets. Step 1 includes identifying optical imaging markers that are predictors of -omic markers. Step 2 includes validating optical imaging markers using known-omic characteristics such as results from gene pathway analysis. Step 3 includes developing imaging technologies to acquire the optimal imaging markers in vivo for point-of-care tissue subtyping, phenotyping, diagnosis, or pathology enhancement.

To perform any of methods 1-4, the -omics data may be acquired in one or more of number of methods. These methods include:
1. Existing spatial-omics technology (transcriptomics, proteomics, genomics),
2. Laser capture microdissection followed by standard (non-spatial)-omics analysis,
3. Mechanical dissection followed by standard (non-spatial)-omics analysis, and
4. Single cell sequencing.

Optical imaging data may be acquired using a variety of techniques. These include:
1. Autofluorescent imaging (including multiphoton imaging)
2. Fluorescence lifetime imaging
3. Raman-based techniques (CARS, SRS, etc.)
4. Hyperspectral imaging
5. Polarization imaging
6. Other optical imaging modalities that provide contrast to biochemical, functional, or microstructural tissue characteristics.

The resulting-omics and optical imaging datasets may be coregistered pixel by pixel (tissue-by-tissue, or cell-by-cell for non-pixel-based methods). Analysis is conducted to identify imaging markers that are predictors of -omics characteristics. Analysis may include correlation analysis, principal component analysis, machine learning techniques, among other classification and clustering methods. The result is a set of optical imaging markers that have high accuracy in predicting specific-omics characteristics.

Embodiments have applications including the following.
1. Imaging marker validation: Confirming that collected imaging markers recapitulate abnormalities caused by disease by demonstrating correlation with -omics markers.
2. Develop in vivo imaging technology: Tunable imaging technology may be developed to acquire the optimal imaging markers rapidly and potentially in vivo. This may be used for point-of-care tissue subtyping, estimation of disease prognosis (e.g., cancer metastatic potential), or evaluation of overall tissue health.
3. Pathology enhancement: The identified optimal imaging markers may be used for rapid nondestructive estimation of genetic, transcriptomic, or proteomic markers to enhance pathological examination of tissues by reducing processing time.
4. Cell or tissue phenotyping: The technology may be used for rapid phenotyping of cells or tissues (potentially in vivo), assisting with disease diagnosis and management by informing the therapeutic approach.

### 1.2 Impact

Embodiments disclosed herein are based on improved understanding of how sNF-pNET microenvironments modify tumor metastasis and what cell pathways are involved in these variations. In embodiments, technical and computational methods are part of imaging methods capable of detecting these variations. The determination of autofluorescent image features that model genetic variations in sNF-pNETs may support the creation of tissue-preserving optical imaging methods for determining patient prognosis that may be used in conjunction with current pathology or, in the long-term, find its use in the clinical setting.

### 1.3 Significance

Neuroendocrine neoplasms (NENs) are a broad category of cancerous masses defined by their composition of cells expressing neuroendocrine markers, such as neuroendocrine secretory/membrane proteins, hormones, and transcription factors. NENs are classified by their site of origin, hormone functional status (i.e., functional when cells express hormones, non-functional otherwise), and the differentiation and proliferative rate of cells within the mass. Neuroendocrine tumors (NETs) represent a class of well-differentiated NENs that have shown a steady increase in incidence over the last five decades, with lung, GI tract and pancreas the most common sites of occurrence. Improvements in early detection and treatment has resulted in the increased survival rate for all NETs yet significant gaps in our understanding of this disease have contributed to controversy over ever-evolving treatment guidelines.

Pancreatic neuroendocrine tumors (pNETs) are a subset of NETs that pose a complex challenge in clinical decision-making due to their rare incidence compounding the difficult process of establishing accurate classification and staging tools for this highly heterogeneous disease. NENs, including pNETs, are considered malignant by default and surgical resection is recommended for all functional and localized non-functional pNETs (NF-pNETs), as this remains the only potentially curative treatment. Because the risk of malignancy increases with functional status and greater pNET size, more conservative ‘watch-and-wait’ or lymph node-sparing resection approaches for NF-pNETs<2 cm is suggested in situations where the avoidance of surgical risk is desired. However, conflicting findings on the malignant risk of small NF-pNETs (sNF-pNETs) suggests further pre-operative characterization of tumors within this group are warranted. For instance, reports of the three-fold increase in overall patient survival following resection of NF-pNETs and the high risk of malignancy for tumors within this class conflicts with aggregate data of pNET small-needle biopsies and incidentally found stage 1 NF-pNETs showing that a majority are non-malignant. With 81% of all pNETs presenting as nonfunctional, and sNF-pNETs comprising the highest incidence NF-pNETs there is a clear need to improve our ability to characterize the biology sNF-pNETs to make accurate prognoses and facilitate personalized medical treatment.

Advances in CT, MRI, and endoscopic ultrasound have improved the rate of detection and accuracy of localizing NF-pNETs but these modalities lack in their ability to provide insight into much of the biological behavior of the tumors. Current practices in tumor grading rely on cell differentiation status, Ki-67 indexing and mitotic count determined through pathology. Several studies have shown pitfalls in this process, such as the Ki-67 index underestimating tumor grade or changing through the course of the disease lifetime. Furthermore, the introduction of non-neuroendocrine neuroendocrine neoplasms to emphasize the existence of mixed tumors with different grades in the 2017 World Health Organization classification of pNETs suggests that the sampling bias introduced by needle biopsy or incomplete resection can severely confound the accurate interpretation of tumor biology by pathology.

While proper tumor grading poses as a challenge to pathologists, the inclusion of molecular and clinical information has shown to improve classification accuracy. Several biomarkers and genomic variations in pNETs have been successfully used to correctly discern between distinct changes in tumor behavior. While these methods of classification warrant further study before widespread use, they exemplify the utility of measuring molecular-specific information of pNETs for pre-operative decision making. For instance, the combination of miRNA and mRNA transcription analysis using samples consisting predominantly of NF-pNETs and insulinomas has determined three human pNET subtypes, termed islet/insulinoma, intermediate (INT) and metastasis-like primary (MLP). NF-pNETs were found to be either the highly metastatic MLP subtype, or the INT subtype with mixed metastatic risk. Based on current pNET grading standards each subtype, including the non-metastatic IT subtype, were found to contain grade 2 tumors, further exemplifying the heterogeneity of this disease and ambiguity of standard grading methods. Interestingly, the pNET molecular subtypes were also shown to have distinct metabolic variations. Compared to the other subtypes, MLP pNETs exhibited a lower dependence on the pyruvate cycling metabolism seen in pancreatic islet β cells, suggesting a lower cellular concentration of NADPH that is regulated for this pathway.

### 1.4 Spatial Transcriptomics

Spatially resolved genetic and transcriptional profiling is an emerging set of methods that has quickly found a footing in cancer research. The retention of spatial organization allows for the study of tumor heterogeneity and has been used to distinguish different multicellular communities with distinct treatment responses in pancreatic cancer. While a plethora of techniques exist, review of specific spatial transcriptomic technology may be confined to resources available at the University of Arizona.

Digital Spatial Profiling (DSP) provides a multiplexed readout of RNA or protein expression in formalin-fixed, paraffin-embedded (FFPE) samples through the binding of oligonucleotide tags attached to antibodies or RNA probes. Once bound, the oligonucleotides are removed with a photocleaving process in regions of interest (ROIs) created by the user over an immunohistochemical (IHC) image of the sample. The IHC staining is intended to identify tissue features of interest to guide the sampling process. The cleaved oligo tags from ROIs are pooled into separate batches and hybridized to fluorescent barcodes that act as labels during an imaging process that directly counts the number of bound molecules. This process avoids the need of amplification used in traditional RNA sequencing, which can introduce bias in the results.

The 10× Genomics Visium platform uses a similar oligonucleotide tagging process. In this case, oligo tags are arranged in regions of 55 μm diameter for a total of 5,000 regions per sample. Each region has a unique barcode that is used to identify the spatial origin of whole-transcriptome readouts.

Neither of these platforms are currently capable of measuring miRNA expression in tissue samples. Early methods of spatially resolved transcriptomics successfully applied laser-capture microdissection (LCM) to remove selected portions of tissue that are catalogued prior to sequencing. LCM has been used to successfully isolate objects ranging in size from tumor regions to subcellular components. The basic components used in LCM are a traditional microscope with camera attachment, UV laser, laser control unit, and digital display for real-time image viewing. Current LCM systems require minimal operator training and allow for precise laser control to cut tissue regions which are dropped via gravity into individual sample tubes. The inclusion of miRNA sequencing is vital for the expansion of our understanding of the biological significance of established pNET molecular subtypes.

### 1.5 Autofluorescent Imaging in Biology and Medicine

Autofluorescence (AF) imaging is the process of detecting fluorescence emitted from endogenous fluorophores following light excitation of biologic specimens. These fluorescent molecules have distinct excitation and emission spectra, and vary in their location and activity within cells, connecting them to distinct processes that can change through the lifetime of a cell. Variations in autofluorescence arise from differences in fluorophore concentration, conformational state, and/or spatial distribution which change based on different intrinsic and extrinsic factors such as genetic expression or disease state. These variations in autofluorescence have been leveraged to distinguish between tissue and cell types, which has extended to numerous applications in cancer research. Because autofluorescent imaging does not require additives or fixation of specimens, it may be used to capture real-time information of tissue physiologic state and has been applied in endoscopy as an ‘optical biopsy’ tool to distinguish lesions in vivo.

Collection of autofluorescence may be achieved through either single or multi-photon excitation of fluorophores, each method with its own strengths. Single-photon excitation (SPE) may be used in widefield imaging to collect fluorescence from a large area of the specimen, expediting acquisition time. Widefield imaging results in the collection of both in-focus and out-of-focus fluorescence causing a reduction in image contrast. Multi-photon microscopy (MPM) is based on the two-photon effect (2PEF), where a high photon density can result in the near-simultaneous absorption of two photons by a single fluorophore resulting in nonlinear molecular excitation and photon emission of a shorter wavelength. Another source of MPM image contrast is through second-harmonic generation (SHG), a light-scattering phenomenon from non-centrosymmetric structures which is predominantly collagen in tissue. SHG is strongly influenced by collagen morphology, where non-random molecular organization enhances the signal generated via SHG. MPM is an inherently high-resolution imaging technique due to the sub-femtoliter volume of space that the 2PE occurs within. MPM also utilizes light of higher wavelengths compared to SPE due to the additive excitation of the absorbed photons. These properties of MPM reduce the likelihood of photobleaching fluorophores outside the volume of excitation and allow for volumetric imaging of tissue at greater depths, making it widely popular for imaging biologic samples. Unfortunately, the laser-scanning time required to acquire a full image with MPM may be prohibitive towards the collection of multiple wavelength channels. SPE can therefore be used to define significant autofluorescence features of tissue that may be probed further with MPM.

Collection of tissue autofluorescence signal over a broad band of wavelengths is possible with hyperspectral imaging (HSI), which is the process of collecting both spectral and spatial information within an image. During HSI, spectral information is collected in the form of photon intensity over a range of wavelengths at each pixel within the two-dimensional pixel array of the detector. The spectral information combined with the location of its detection on the two-dimensional detector is used to generate a three-dimensional dataset known as a hypercube. HSI using tissue autofluorescence can create a rich dataset that provides insight into the abundance of distinct fluorophores within cells using the process of spectral unmixing which decomposes the measured spectrum within a pixel into its constituent endmembers.

### 1.6 Connections Between Cellular Pathways, Autofluorescence, and Second-Harmonic Generation

Cellular senescence is a multifunctional process that represents a halt in cell proliferation and a transition in cell phenotype which can arise from numerous effectors on multiple cell signaling pathways. Cell autofluorescence has been shown to increase with a transition into senescence which is believed to be related to an increase in cytoplasm granulation and lipofuscin aggregation. The variation in metabolism between pNET molecular subtypes seems to mimic the Warburg effect, a common finding in cancer cells where a shift to aerobic glycolysis occurs through a variety of possible genetic alterations. With this shift in cell metabolism, a change in the concentration of co-enzymes NADH, NADPH and FAD that are involved in these metabolic pathways occurs. The distinct fluorescence spectra of NAD (P) H and FAD facilitate a quantitative means of determining variations in cell metabolic status.

Moving outside the cell, changes in the tumor microenvironment have been shown to modify collagen morphology with highly aggressive tumors exhibiting distinct collagen morphology changes compared to low-risk tumors. Variations in tumor extracellular signaling have been shown to cause increased disorganization of collagen and an increased ratio of elastin vs collagen in neoplastic vs normal tissue. Similar alterations in pNET stroma have been used to create convolution neural networks that can accurately predict pNET malignancy based on tumor morphology, showing its utility as a feature included in predictive models. While a connection between stromal modification and pancreatic neoplasm genetics has been established, a direct comparison of local stromal variation and genetic expression within a tumor mass has not yet been done. The combination of SHG imaging with spatial transcriptomics may explain if distinct cell mutations or patterns in gene expression result in these high-risk stromal morphologies.

### 1.7 Microscopy Images

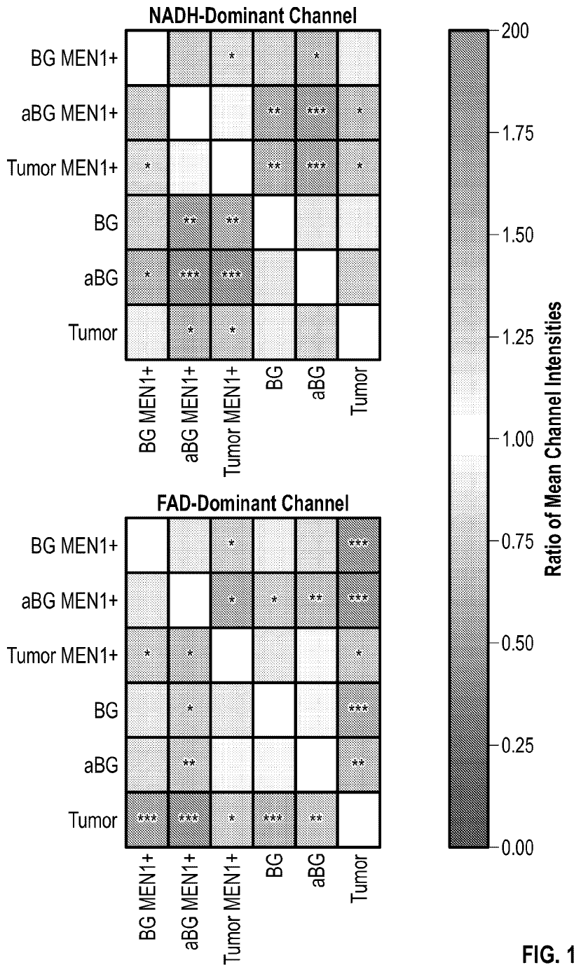

FIG. 1 includes Heatmaps showing comparisons of relative abundance of MPM fluorescence channel intensities. MPM channels are indicated by heatmap title. Tile color indicates the ratio of mean normalized pixel values from the aggregate regions of interest (ROIs) of the respective tissue types that have been separated between patients with and without MEN1 mutations (MEN1+ indicates the presence of the genetic mutation). BG=Brunner's gland, a naturally occurring gland in the small intestine. aBG=abnormal Brunner's gland, where abnormal classification was determined based on unusual PanCK staining and proximity of this tissue to DGAST tumor regions. Asterisks show statistical significance of difference between groups determined using independent t-test, *=p<0.05, **=p<0.01, ***=p<0.001.

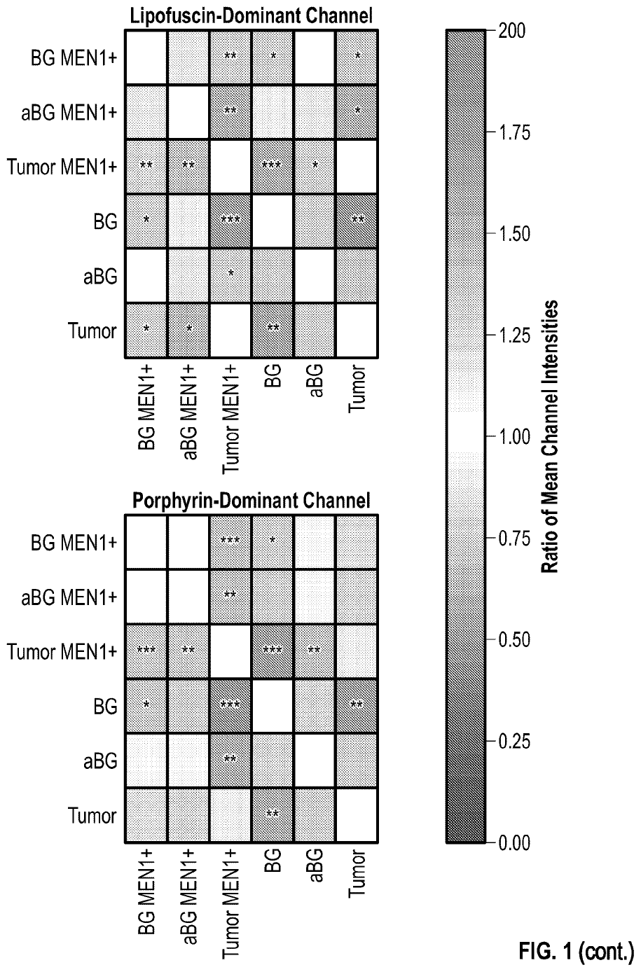

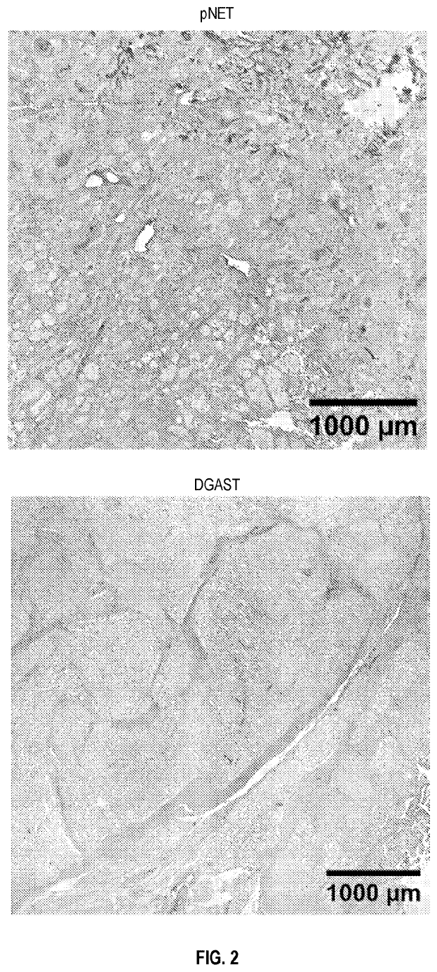

FIG. 2 includes Example images of PNET and DGAST acquired using MPM, both imaged with parameters set to acquire fluorescence predominantly from lipofuscin and lipofuscin-like fluorophores (i.e., Lipofuscin-dominant channel parameters).

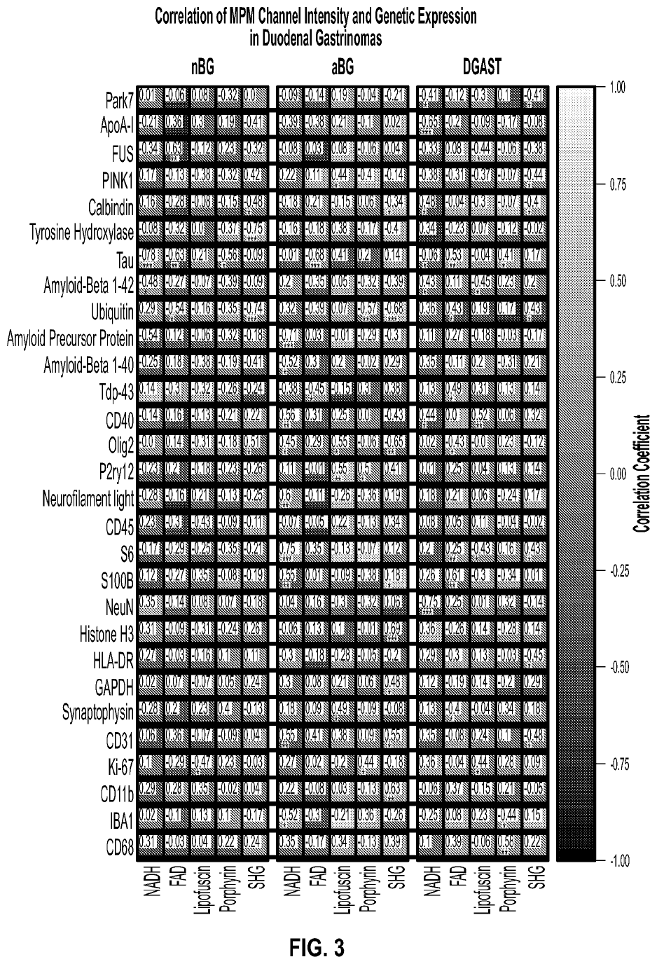

FIG. 3 illustrates correlation heatmaps between genetic expression and MPM image channel intensities. Values in each matrix element shows the linear regression coefficient of DSP genetic expression and MPM channel intensity values. Asterisks show linear regression p-values, where *=p<0.05, **=p<0.01, ***=p<0.001. nBG/aBG=normal/abnormal Brunner's gland, DGAST=duodenal gastrinoma.

Gastrinomas are functional NETs that arise most commonly in the duodenum (DGAST) or pancreas. Multiple endocrine neoplasia-1 (MEN1) syndrome is a hereditary mutation of the genetic sequence for menin, a tumor suppressor protein. Alterations in menin secondary to MEN1 syndrome has been linked to a high incidence of NETs and many sporadic pNETs have been found with MEN1 mutations not found in germline cells of the patient. MPM has been used to characterize differences in the autofluorescence abundances of tissue types (e.g., normal gland/structural tissues vs tumor) found in human DGAST samples. We have shown that MPM detects autofluorescence of numerous wavelengths that varies significantly between DGAST and surrounding normal tissue. Classification of DGAST tissue based on patient MEN1 status (FIG. 1) suggests there is a measurable difference of autofluorescence in NETs that is influenced based on genetic expression. MPM imaging of both DGAST and pNET samples using the same acquisition parameters (FIG. 2) shows pNETs produce adequate autofluorescence signal for analysis. Areas of interest (AOIs) from DSP spatial transcriptomics has been correlated with ROIs drawn from MPM images of DGAST tissue serial sections (FIG. 3). Results show variations in the degree of correlation between MPM channel intensity and genetic expression. This suggests spectrally resolved autofluorescent imaging over a broad wavelength range would improve the determination of relationship between spectral features and variations in genetic expression. The change in autofluorescence correlation with genetic expression between tissue types supports the use of spatial transcriptomics for developing a model of gene expression and autofluorescence, as bulk tissue sequencing would make distinct connections between image measurements and tumor biology unfeasible. An early regression model using K-Nearest Neighbors (KNN) shows decent predictive accuracy of genetic expression in DGASTs based on autofluorescence variations (Table 1). Again, variations in KNN regression accuracy with inclusion of different MPM channels suggests a wide spectrum of tissue autofluorescence data would provide the best means of accurately modeling variations in genetic expression.

Table 1 shows results of KNN regression performed with Scikit Python package. Highest test accuracies were found through the iteration of all possible combinations of the continuous data from MPM channel intensities that were then used as training samples for the prediction of genetic expression values for each gene measured with DSP. N neighbors was set to six with uniform weighting.

TABLE 1

| MPM channel(s) used | genetic | test |
|---|---|---|
| in KNN regression | marker | accuracy (%) |
| FAD | PARK7 | 89.58 |
| NADH, FAD, lipofuscin | CD11b | 87.43 |
| NADH, FAD, lipofuscin | CD31 | 86.78 |
| FAD, lipofuscin, SHG | FUS | 86.31 |
| NADH | Tyrosine | 86.29 |
| | Hydroxylase | |
| FAD, lipofuscin | CD45 | 84.11 |
| NADH, FAD, lipofuscin, | CD31 | 82.68 |
| SHG | | |
| SHG | Histone H3 | 82.67 |

### 1.8 Sample Description

Methods disclosed herein my utilize FFPE NF-pNET sample. The samples may be less than two centimeters in size to address the primary research goal of determining the biologic and optical markers for tumors of the sNF-pNET class that pose a greater metastatic risk. The samples may include at least 12 samples of both metastatic and non-metastatic primary tumors so that comparisons may be made between the groups. Method disclosed herein may include collecting three FFPE serial section slides from each patient tumor sample, two for each sequencing step and one as a backup for errors in the staining or sequencing stages. FFPE slides of adjacent normal pancreatic tissue, serum samples and clinical information, including pathology reports may be collected for each patient sample. Methods disclosed herein may include using the samples for further biomarker screening, determination of metastatic status of the tumor, and comparison against the original pathology interpretation. Said methods may include bulk sequencing adjacent normal pancreatic tissue for use as a baseline comparison against the corresponding pNET tumor sample to determine changes in genomic and transcriptomic regulation.

### Method 1

In the aforementioned method 1, said laser-capture microdissection (LCM) may be performed using a Leica LMD6500. FFPE tissue may be stained with hematoxylin and eosin (H&E) to facilitate the designation of normal pancreas vs tumor. Depending on tumor morphology, dissection may be used to either separate distinct tumor regions within the sample slide or to dissect large tumors (e.g., when only a single large tumor is present) in a grid array pattern. In embodiments, dissected tissue is collected in separate micropipette vials labeled for the region from which the tissue was removed in the original H&E image captured by the microscope.

For this work, LCM has several advantages for miRNA analysis over traditional bulk tissue processing. Method 1 may include removing extraneous tissue surrounding the regions of interest, which reduces miRNA readout noise, allowing for sensitive comparisons of regulation changes between samples and the accurate determination of pNET subtype based on miRNA expression. Method 1 may include separation of tumor regions, or the dissection of large tumors into smaller sections, which facilitates the investigation of simultaneous tumor types existing within the same region or mass. Furthermore, images collected during LCM may be used to correlate miRNA expression to the spatial transcriptomic data collected as a separate aim in this project.

In embodiments disclosed herein, tissue processing and miRNA analysis provides a readout of up to hundreds of human miRNAs per sample. Data may be handled using the companion nSolver software, which performs data import, quality control, normalization, export and analysis. In embodiments, relevant biological correlations of genetic expression are be determined using nonnegative matrix factorization (NMF). NMF is a means of reducing the high-dimensional genetic dataset into sets of metagenes that facilitates sample clustering based on expression characteristics. Method 1 may include visualizing up/down regulated miRNA expression using heatmaps, which are a common form of interpreting transcriptomic data. Method 1 may include grouping samples based on initial classification of metastatic/non-metastatic to determine if trends exist between clinical outcome and NMF clustering. In embodiments, normalized raw data of miRNA expression is exported in .csv format to be included in the full dataset of transcriptomic and optical measurements.

In embodiments of method 1, NMF of miRNA sequencing data is produces clustered data, as this dimension reduction technique has an inherent clustering property. These clusters may present ‘metagenes’ that may be used to classify the NF-pNET samples. Classification using this process may produce subtype groups like those found using a similar methodology. NF-pNET groupings may have clinical outcomes and tumor grades that correlate with their classification which would indicate good agreement with prior establishment of these subtypes.

### Method 2

Method 2 may include transferring FFPE tissue to a proprietary 10× Genomics cassette slide. Samples may then be deparaffinized and undergo IHC staining using a combination of DAPI and fluorescently labeled anti-PanCK, synaptophysin, and Ki-67. Prior to sequencing, fluorescent images may be acquired for later registration with the sequencing readouts that are spatially resolved by their unique barcode identifiers.

As a nuclear stain, DAPI provides general tissue morphology information and insight into the specificity of Ki-67 staining. PanCK is predominantly expressed by epithelial cells, providing tissue morphology information with staining, and as a common marker for non-neuroendocrine pancreatic cancers, can provide insight into potential regions of mixed tumor types. While both chromogranin A and synaptophysin (SYP) are common markers of neuroendocrine tumors (NETs), a comparative study has shown anti-SYP to stain NETs stronger and more reliably.

In embodiments, staining, e.g., SYP staining, is used to confirm diagnosis of pNET and delineate regions of NET within the sample. Ki-67 staining may be used to determine the Ki-67 index per standard protocol, Nₚ/N_{F}, to compare this standard of tumor grading against the full set of transcriptomic data and the clinical report associated with the sample. Nₚ is the total number of positively stained tumor cells. N_{F} is the total number of tumor cells within image field.

Method 2 may include processing of raw data and registration of the IHC fluorescence images with the sequencing data, e.g., using the 10× Space Ranger software. Expression data may be normalized prior to dimension reduction using principal component analysis (PCA) where gene expression features are used as the PCA features. After PCA, barcode regions that contained genomic readings above threshold value may be clustered based on expression levels without consideration of spatial information. In embodiments, T-distributed Stochastic Neighbor Embedding (t-SNE) is used to create 2-D representations of the clustered regions, each of which may be given a distinct classification for later analysis. Method 2 may include determining differentially expressed features between clusters, e.g., based on the log 2 fold-change with an adjusted p-value (e.g., less than 0.05) denoting a significant variation between clusters. The method may include creating plots projected over the 2D fluorescence image showing the total genes per spot, number of unique molecular identifiers per spot, cluster classification of each spot, and spots containing selected genes of interest.

MEN1 mutations are the most common genetic variation found in pNETs for both sporadic cases and those associated with germline mutations. This is expected to result in a lower expression of menin in NET regions and to be associated with metastatic status, which may vary depending on concurrent genetic mutations. Conflicting findings of prognosis based on similar mutations suggests that regional differences within the pNET mass, or the occurrence of mixed tumor, may be found using spatial transcriptomics that could begin to elucidate this phenomenon.

### Method 3

Prior to the spatial sequencing performed in Specific Aim 2, NF-pNET samples adhered to the 10× Visium slides may be imaged using non-destructive optical techniques so that direct comparisons may be made between imaging and sequencing data. Initial autofluorescence measurements may be made using an IX71 inverted Olympus microscope with an IMEC SNAPSCAN VNIR hyperspectral system. The hyperspectral system has a spectral range of 470-900 nm sampling 150 bands yielding a spectral resolution of ~2.8 nm. Each filter has a collimated full-width at half maximum of 10-15 nm and the system uses a 1088×2048 pixel sensor with a 10-bit dynamic range. A 20×0.75 NA APO objective may be used to image samples, providing a spatial resolution of approximately 0.56±0.18 microns over the range of emission wavelengths. Higher-magnification objective, e.g., 40× or 60× may be used for imaging. A 150 W Xenon arc lamp may be used as the excitation source.

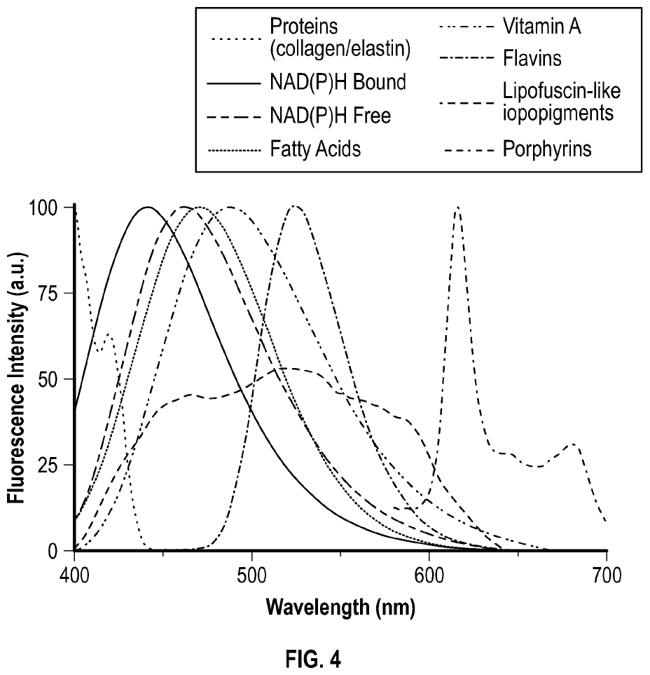

The excitation light spectrum may be tuned using a filter wheel set with bandpass interference filters. Interference filters can achieve higher transmission compared to absorption filters and are most cost-effective over a tunable filter system. Ideally, a set of at least 30 filters with a 10 nm bandpass range may be used to generate excitation wavelengths over the range of 400-700 nm which is the predominant excitation band for endogenous fluorophores, as shown in FIG. 4. FIG. 4 is a fluorescence spectrum of primary fluorophores found in human tissue and their signal intensity over a broad band of excitation wavelengths.

This would result in an excitation-emission matrix of 30 excitation bands by 150 emission bands for each pixel on the 1088×2048 sensor for each session of image acquisition. This dataset would expand depending on the number of images required to capture autofluorescence from the entire pNET tumor at a maximum of the 6.5×6.5 mm area of the Visium tissue slides. Like the miRNA sequence data, multi-layer NMF (MLNMF) may be used for spectral unmixing and dimension reduction of the hyperspectral data. This may be done using the Python scikit-learn package NMF implementation combined with a multilayer decomposition approach to determine endmembers (i.e., fluorophores) contributing to the separate pixel values of each image. NMF was chosen over a PCA approach towards dimension reduction due to its improved performance in extracting features from continuous image datasets.

A Zeiss LSM 880 microscope with a tunable MaiTai HP titanium: sapphire light source and tunable detector will then be used to collect SHG images of the pNET samples. 2PEF images may be collected based on the excitation/emission bands for the major autofluorescence features determined using HSI. 2PEF images may be collected as Z-stacks using an automated motorized stage to perform ‘tile-scanning’ to collect full-sample images of pNET tumors. MPM image data may be in the form of N 2-D matrices containing 16-bit pixel intensity measurements, where N is the number of Z-stacks which is dependent on the obtainable signal through the thickness of each specimen (i.e., the maximum imaging depth) and the step size between each image of the Z-stack. Analysis of collagen morphology may be done using the ImageJ Fiji processing package to extract features such as orientation, arrangement, and density from the SHG images.

ImageJ and Python 3.0 may be used to manipulate and analyze MPM data matrices. First, elements of the MPM matrices may be classified based on t-SNE clustering. In the occurrence that Specific Aim 2 becomes unfeasible, elements will instead be classified based on H&E/IHC staining. ImageJ may be used for image segmentation and the creation of ROIs which will provide the location of matrix elements for each cell cluster and/or tissue type. Image features, such as the relative abundance of fluorescence from each channel and Haralick texture features will then be extracted using Python.

MEN1 mutations have been associated with variations in cellular autofluorescence, senescence, proliferation, and tissue morphology. Each of these variations are expected to be detectable with hyperspectral autofluorescence and/or MPM imaging, either through distinct image texture features or differences in the relative intensities of autofluorescence from fluorophores associated with changes in cell physiology such as metabolism and senescence. Lipofuscin and lipofuscin-like lipopigments are strongly fluorescent compounds with concentrations associated with oxidative stress, cellular senescence, and mTOR pathway function. Due to the relatively broad excitation and emission bands of lipofuscin and its association with several variations seen in cancer, this fluorophore may contribute to significant imaging features distinguishing between cell clusters and differentially-expressing tumors. It is expected that cell types clustered based on expression data may exhibit different autofluorescence spectra. Unmixing of these spectra may show that these variations are a result of differing fluorophore ratios or differences in the presence of detectable fluorophores appearing in some cell types and not others. Image classifiers, such as a convolutional neural network, trained using image properties like texture features and fluorescent intensities along with corresponding expression data of the image region are expected to provide outputs predicting genetic variations when given the autofluorescence spectrum of NF-pNET tissue.

### 2 Validation of Label-Free Optical Imaging Markers of Pancreatic Cancer Using Spatial Transcriptomics

Label-free biomedical imaging represents a range of powerful technologies used to visualize natural sources of biological contrast. Label-free techniques such as autofluorescence and fluorescence lifetime imaging measure contrast produced by various cellular products and provide high sensitivity for detecting tissue changes that occur with disease onset. However, a major limitation of these modalities, and many label-free modalities broadly, is the lack of robust validation methods that confirm signal specificity. Moreover, existing approaches are limited to assessing correlations and fail to provide mechanistic information into pathological events.

Spatially resolved gene sequencing methods (e.g., spatial transcriptomics) are a powerful tool to gain detailed biological insight into tissue properties by creating 2-D maps of variations in gene expression that influence tissue properties. Thus, these techniques represent an avenue for validation of label-free imaging markers through the examination of how label-free image features correspond to gene expression.

Toward this aim, we performed autofluorescence and fluorescence lifetime imaging on tissue specimens from four patients presenting with pancreatic neuroendocrine tumors. We then performed spatial transcriptome sequencing on serial tissue sections to measure transcriptome-wide signatures. We assessed imaging biomarkers related to cellular metabolism, vasculature, and extracellular matrix properties. After registering the label-free images to the transcriptomic signatures, we performed k-means clustering, and assessed the correlation between imaging markers and differentially expressed genes associated with tissue properties of interest. Specifically, we examine correlations between gene expression and established optical biomarkers (e.g., optical redox ratio), along with identifying other potential connections between label-free optics and cellular genetics. The results show that spatial transcriptomics may be used as an effective validation tool for label-free imaging markers, while simultaneously providing additional biological insight to improve the specificity of imaging studies.

### 2.1 Introduction

Label-free biomedical imaging broadly constitutes a range of imaging technologies that exploit natural contrast mechanisms in the body to visualize tissue and glean information about underlying biological processes.¹ Many subtypes of label-free imaging exist, spanning a range of resolutions, fields of view, and contrast mechanisms. Notable examples include techniques sensitive to tissue microstructure such as optical coherence tomography and polarization-sensitive imaging.^{2,3} Other techniques, such as those based on spectral imaging, are sensitive to biomolecules with high optical absorption, including hemoglobin and melanin.⁴

Tissue fluorescence is also a powerful source of contrast, as many natural biomolecules fluoresce-autofluorescence microscopy aims to measure these natural fluorescent markers, which include collagen, metabolic co-factors such as NADH and FAD, lipopigments, and porphyrins, among others.⁵ When using multiphoton excitation to probe these markers, harmonic generation events are also stimulated; one of the most widely utilized is second harmonic generation to map collagen networks.^{6,7}

As such, multiphoton microscopy (MPM) represents a powerful technique to simultaneously collect tissue autofluorescence and harmonic generation, providing exquisite maps of label-free biomarkers. Fluorescence lifetime imaging (FLIm) is a modality of fluorescence imaging that measures the lifetime of the fluorescent emission in addition to the abundance of a fluorophore.^{8,9} This enables one to measure label-free biomarkers with more specificity, such as the relative intensities of NADH to FAD, to evaluate the “optical redox ratio” as a measure of tissue or cellular metabolism.¹⁰

In the context of imaging, “imaging markers” refers to any quantitative or qualitative descriptor derived from an image. Some examples include average brightness, morphological or texture features, among many others. Identifying and evaluating imaging markers represents a broad field of image science with the overall goal of determining repeatable and robust measures of image content that represent a biological or clinical feature of interest.¹¹ Such imaging techniques carry significant promise for a variety of clinical applications, including disease diagnosis, surgical guidance, and digital pathology.

One major challenge in label-free imaging-particularly relating to tissue fluorescence—is the need for rigorous validation. In many cases, the specific source of label-free fluorescence can be difficult to precisely identify, as biological fluorophores often have significant spectral overlap.⁵ While computational methods have been developed to unmix overlapping signals,¹² these methods often rely on accurate measurement of ground truth spectra (e.g., spectra of a “pure” material).¹³ Such ground truth measurements generally don't recapitulate true in vivo characteristics or account for significant variability that exists in human tissue. Even when successful, this approach for validation does not provide any information about why a contrast source may be varied between two experiment groups, which severely limits the generalization of the results to other cohorts, tissues, or diseases.

Other methods for validation include histological staining and microscopy, often used as the gold standard for identifying tissue changes and confirming disease diagnosis.^{14,15} These approaches include chromogenic staining (e.g., Hematoxylin and Eosin, or H&E) and immunofluorescent staining methods. These methods allow one to determine specific cell types, and occasionally sub-populations using multiplexed antibody staining or in-situ hybridization. While valuable, limitations in this approach lie in the spectral properties of the antibody fluorophore and imaging system, typically allowing for a maximum of seven targets at a time. Therefore, a limited range of information may be elucidated about the tissue using a single round of staining, requiring further cost and time for additional measurements to be made. In addition, quantification of fluorescence microscopy images can be highly challenging and error-prone, and most often it is used for binary labeling of tissue types (e.g., expression is assessed as positive or negative).¹⁶ Therefore, these approaches can be used for validation only in that it allows one to correlate label-free images and subsequently derived markers with certain tissue types or classes, limiting one's ability to understand mechanistically what is giving rise to the observed characteristics. Ultimately, this too reduces the generalizability of observations beyond a specific study.

Moreover, these approaches are not ideal for characterizing disease subtypes or heterogeneous tissues, where the expression of a single marker does not fully describe the overall tissue behavior. One clear example is in the case of different grades of pancreatic neuroendocrine tumors (PNETs), where tumors may broadly express a neuroendocrine marker such as chromogranin A (which would be used for classification via fluorescence microscopy),¹⁷ yet demonstrate vastly different behavior. For example, Grade 3 NETs are, by nature, more proliferative and present with a poorer prognostic outlook. These characteristics may be detectable via label-free imaging modalities that are sensitive to metabolic contrast.¹⁸ Therefore, there is a clear gap in developing validation methods that provide a more comprehensive understanding of the underlying tissue biology and potential patient-specific variability that would not be captured using existing validation techniques. Broadly, a more comprehensive approach to validation could enable significant advancements in label-free imaging by enabling a better understanding of how label-free contrast may be regulated by more fundamental biological changes, and how label-free imaging could be better leveraged for biomedical applications.

A promising approach to bridging the gap to more comprehensive validation of label-free imaging is the application of sequencing technologies. The past two decades have seen rapid advancements in sequencing (or “-omic”) technologies that now enable one to rapidly generate a comprehensive map of the entire genome or transcriptome.¹⁹ These approaches, particularly RNA sequencing or transcriptomics, carry great promise for comprehensive validation of label-free imaging markers, as they provide information about gene expression and regulation that dictate downstream tissue characteristics giving rise to light-tissue interactions.

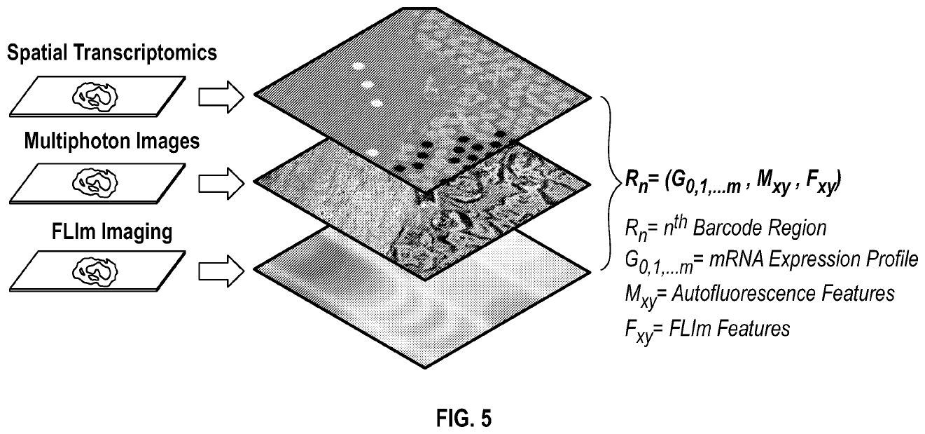

Of the various sequencing technologies available, spatial transcriptomics allows one to generate spatial maps of gene expression.²⁰ Multiple variations of this technology exist, but one example is the 10× Genomics Visium Spatial Gene Expression assay, which maps the entire transcriptome across a 6.5 mm×6.5 mm sample area, with “barcoded” points spaced in a grid with a period of 100 microns.²¹ We have applied this technology to evaluate spatial transcriptomics for validation of label-free imaging markers derived from multiphoton microscopy and fluorescence lifetime imaging. We conducted spatial transcriptome sequencing on four specimens collected from patients with PNETs, then performed MPM and FLIm on serial tissue sections (FIG. 5). We spatially coregistered the Visium and imaging datasets and extracted regions of interest corresponding to each spatial sequencing barcode region, representing a single “pixel.” Imaging markers were then extracted from each region of interest and analyzed to determine the degree of correlation between imaging markers and gene expression.

We also performed clustering of the transcriptomic dataset and showed that multiple subtypes of tumor exist in our patient cohort, which provides compelling validation of the heterogeneity observed in our label-free images. Ultimately, we have demonstrated that spatial transcriptome sequencing data may be coregistered at the pixel-level with label-free imaging modalities, paving the way to developing advanced validation methods using this technology.

### 2.2 Methods

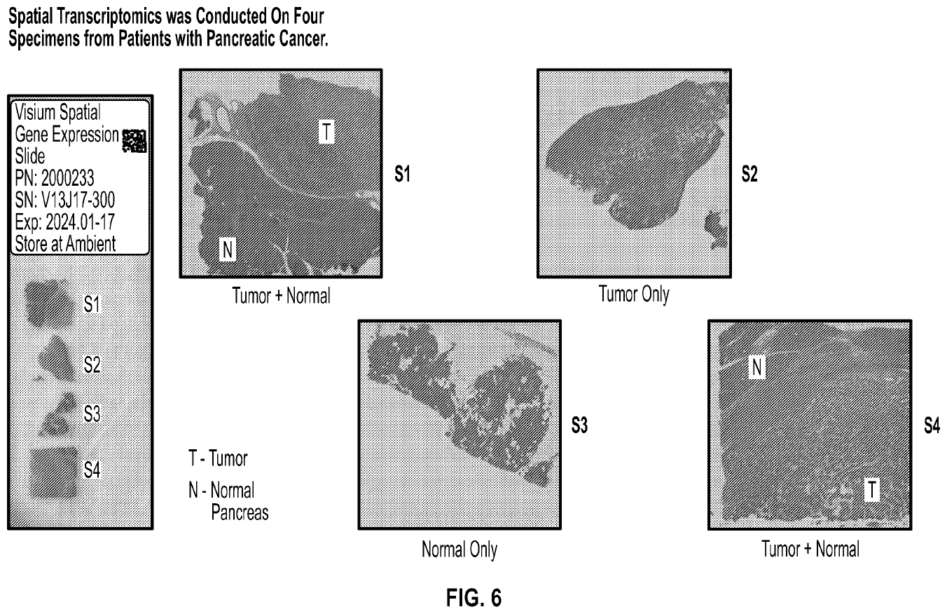

This section describes four patient specimens collected from patients diagnosed with pancreatic neuroendocrine tumors (FIG. 6). The specimens are formalin-fixed paraffin-embedded (FFPE) specimens were obtained from the University of Arizona Tissue Acquisition and Cellular/Molecular Analysis Shared Resource. The specimens were sectioned at a thickness of 7 microns onto microscope slides. Three serial sections were used: one was placed on a specific slide used for the Visium spatial transcriptomics, one was used for MPM, and one for FLIm (a quartz slide).

### 2.2.1 Spatial Transcriptomics

Patient samples were deparaffinized and H&E stained following the standard 10× Genomics protocol for FFPE tissue prior to being slide-scanned using a Nikon BioPipline SLIDE system (Nikon, Tokyo, Japan). Spatial transcriptomics was completed using the 10× Genomics' Visium Spatial Gene Expression system (10× Genomics, Pleasanton, CA, USA). Individual libraries were processed using 10× Genomics' Space Ranger count pipeline. Length of the R2 sequences were limited to the first 50 bps. Space Ranger v2.1.0 count pipeline was used to manually align the fiducial frames of the Visium slide to gene expression data. For easier export, the Visium Space Ranger software down-samples the original high-resolution H&E image such that its longest dimension is 2000, resulting in the other dimension being resized based on the image's original aspect ratio. The exported data frame of gene expression counts contains the Visium barcode coordinates from the original, full-resolution, H&E image. Due to the uniform down-sampling of the original image, a single scaling factor is used to match the original barcode coordinates to the dimensions of the exported H&E image, to which the MPM and FLIm images were registered.

### 2.2.2 Multiphoton and Fluorescence Lifetime Imaging

MPM imaging was done using a Zeiss LSM 780 NLO system with an automated 3D translation stage. Tunable MaiTai Ti:Sa laser and GaAsP detectors were used to image in five spectral channels, four channels capturing the signal from tissue autofluorescence, and a second-harmonic generation (SHG) channel. The excitation and detection wavelengths are shown in Table 2, in which MPM image channels referred to by the autofluorescent species, or optical phenomena (SHG), were targeted by the respective excitation and detection parameters of the system.

Each MPM channel is referred to in this manuscript by the endogenous molecule(s) believed to be the primary source of signal when imaging with the spectral parameters in Table 2. 256×256 pixel tile scans were collected in a grid array over the sample and combined with post-processing to form a composite image of the full specimen as it was imaged on the Visium slide. A 20× objective (make and model) was used, providing a square pixel resolution of ~1.28 micron. Z-stacks were acquired for each tile scan to ensure in-focus signal was collected over the full range of the uneven tissue surfaces, with the number of z-stacks adjusted as necessary. Despite z-stacking, uneven brightness within the individual tile scans results in a grid-like artifact in the image mosaics. Post-processing that has been previously described²² was used to balance image brightness between image tiles prior to quantification.

TABLE 2

| | Excitation | Detection |
|---|---|---|
| Channel | wavelengths (nm) | wavelengths (nm) |
| NADH | 700 | 425-465 |
| Porphyrins | 800 | 590-625 |
| Lipofuscin/ | 830 | 550-600 |
| lipopigments | | |
| SHG | 880 | 430-450 |
| FAD | 920 | 475-600 |

Pulse-sampling FLIm was completed using a fiber-based multispectral system that has been previously described.²³ Each of the specimens was positioned ~3 mm away from the fiber tip and a 355 nm Q-switched microchip pulsed laser (pulse duration <0.6 ns, pulse energy >2 μJ, 460 Hz repetition rate, STV-02E-140, TEEM photonics, France) was used to elicit tissue autofluorescence. A single 365 μm core multimode fiber (FG365UEC; Thorlabs, NJ) with a ball lens termination (3 mm working distance) was used to transmit the laser light and collect the autofluorescence signal. Tissue autofluorescence was propagated to dichroic mirrors and filters that were used to create three separate spectral channels with wavelengths of 390/40 nm, 470/28 nm, and 540/50 nm. Tissue autofluorescence was detected by three UV-enhanced Si avalanche photodiodes modules (APD430A2SP1, Thorlabs, NJ) with variable gain and integrated transimpedance amplifiers, and the resulting electrical transient was recorded by a high-speed digitizer (PXIe-5162, National Instruments, Austin, TX). The fiber optic was raster scanned across the samples using a 3-axis translation stage (PROmech LP28, Parker, Charlotte, NC) to generate full-sample fluorescence lifetime images composed of pixels with an area of 200 μm².

Each pixel of the FLIm image includes of the three fluorescence intensity decays derived from the separate spectral channels. Fluorescence decay measurements were background subtracted to remove the signal generated within the fiber probe and deconvolved using the system response function as previously described.²⁴ Images were further thresholded to remove signals below 30 dB.

### 2.2.3 Image Coregistration

Images were manually coregistered using the open-source TrakEM2 ImageJ plugin.2 The down-sampled H&E images matched to the Visium gene expression data were used as the final registration targets due to the original slide-scanned images being prohibitively large to process. Because the down-sampled H&E images are limited to 2000 pixels in the largest dimension, they had to be scaled to dimensions roughly matching that of the label-free images. In the case of registering the MPM images to the Visium data, the down-sampled H&E image was scaled by a factor of three, changing its largest dimension to 6,000 pixels. With each MPM image having dimensions of roughly 5,500 pixels, any differences in scale could be further accounted for during the registration process.

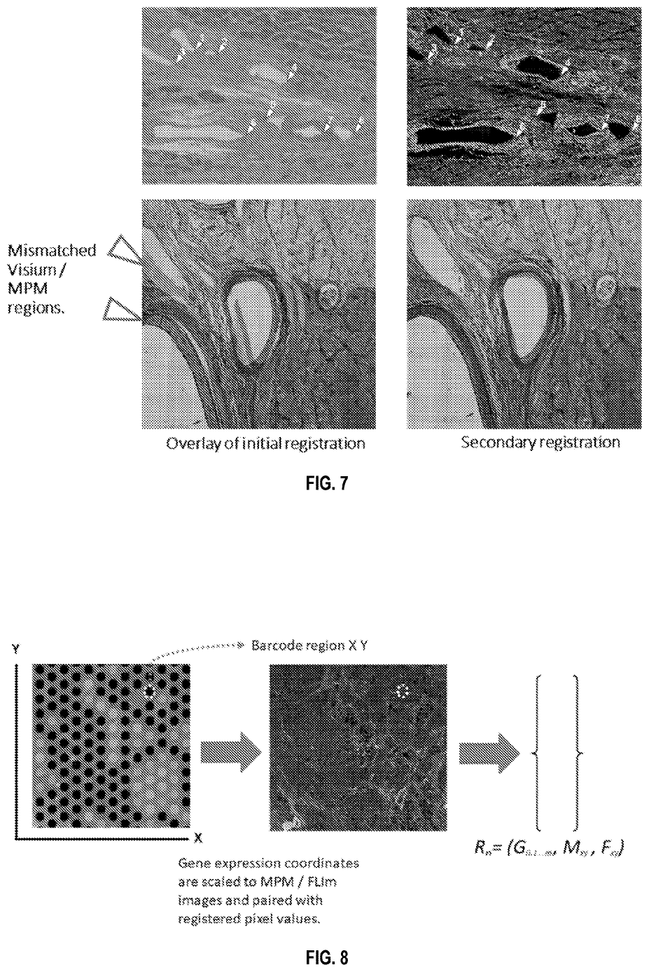

First, multi-channel MPM and FLIm images were registered to a single channel from each respective system. This was done to eliminate image drift between acquisitions, which could arise from variations in spectral propagation through the system's optics, physical movement of the tissue sample, and/or drift of the scanning mechanisms. Registration landmarks were placed on image regions that contained obvious tissue landmarks, such as glands or cell clusters with unique morphology, as shown in FIG. 7A. Multiple rounds of manual landmark placement and registration were necessary to match image contents due to differences in scale and distortion of the tissue during the process of mounting it onto the Visium slide and H&E staining. An initial round of registration through translation, rotation, and isotropic scaling was done to match image dimensions/aspect ratio, and roughly overlay landmarked regions. An affine transformation was then used with newly placed landmarks, repeating as necessary to align image contents (FIG. 7 B). Registered images were then exported in their original 16-bit image format.

### 2.2.4 Quantitative Analysis

Gene expression data was exported into a Python environment as a data frame containing the unique molecular identifier counts for each human mRNA target and the pixel coordinates for the center of each barcode region. The pixel coordinates were scaled to the down-sampled H&E image space and then scaled again to the registered image space. Features from the label-free image were thus matched with the gene expression data through the extraction of regions-of-interest (ROIs) corresponding to the barcode pixel coordinates (FIG. 8). ROIs were scaled by the pixel resolution of the label-free imaging systems to match the ~50 microns diameter of the Visium barcode regions. A threshold was applied to the MPM ROIs to remove background noise (<2000) and saturated pixels (>65530). Autofluorescence abundance was measured by taking the mean value of each MPM ROI. The Haralick texture features of the ROIs were measured in the horizontal, vertical, and diagonal directions and averaged into a single set of the thirteen texture features for each barcode region. Similarly, the intensity of each FLIm channel was measured as the average lifetime value within each ROI.

Clustering of the spatial transcriptomic data was completed using the Loupe browser and K-means clustering was applied with k=7 and all four patient samples pooled together. The selection of k=7 was evaluated by finding the maximum Silhouette score. The results were then visualized by projecting each cluster onto the respective barcode region and superimposing this on an H&E brightfield image to visualize the clusters and spatial relationship. Quantification of the coregistered transcriptomic and imaging dataset was completed by visualizing gene expression for each barcode region against various imaging marker values. Initially, quantification was done by evaluating abundance of each imaging channel. Channels were normalized by dividing by the NADH channel. Visualization of the imaging marker values as probability density distributions was also conducted, where the barcode regions were clustered according to k-means clustering conducted on the gene expression data. Statistical significance of the average value of the distributions was assessed using an independent t-test.

### 2.3 Results and Discussion

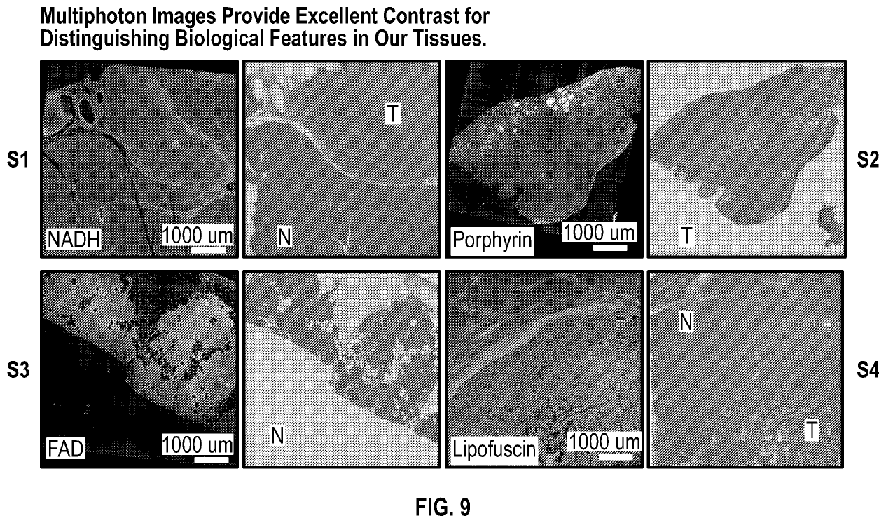

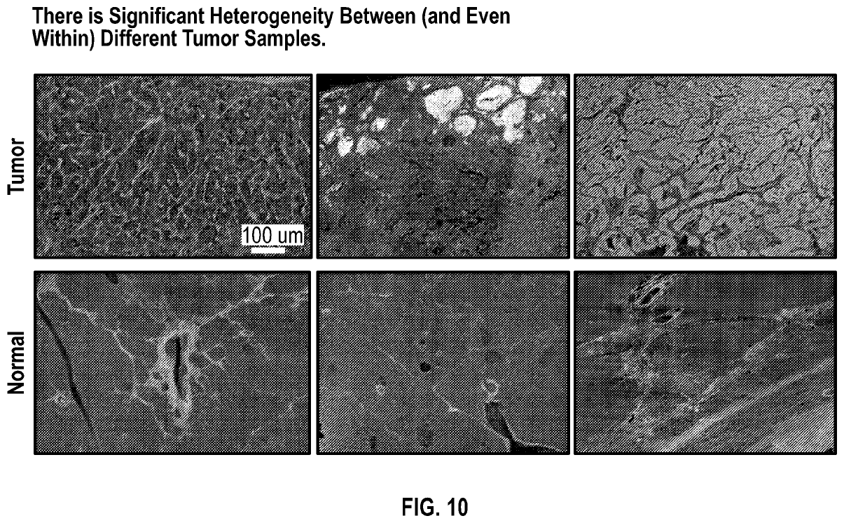

FIG. 9 shows MPM and matched H&E images for each patient sample, with each of the four different channels shown. MPM provides a clear qualitative contrast for tissue features. For example, the porphyrin channel correlates with the abundance of blood. Interestingly, each of the three tumor areas had remarkably different image contrast, particularly when considering spatial variations such as texture (FIG. 10). In contrast, normal tissue had relatively consistent features across all the specimens. There is therefore clear heterogeneity in these tumor specimens—this would pose significant challenges for image feature extraction given that there would be a large variance in the tumor specimens. As such, this highlights the need for more nuanced validation of these samples to capture this heterogeneity.

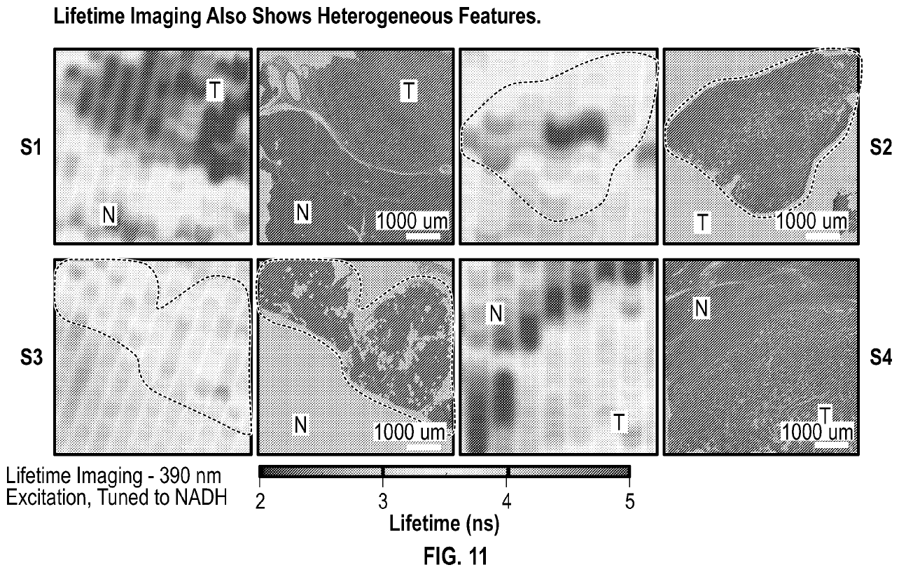

FIG. 11 shows the FLIm images for our specimens in the 390 nm excitation channel. Similar to the MPM images, we see a significant variation in the lifetime of the tumor specimens. For example, S1 has an average lifetime nearing 5 ns, whereas S4 is approximately 3.75 ns. S2 is particularly interesting, as it has heterogeneity within the sample, with the center exhibiting a lifetime of about 5 ns, and the rim closer to 3.5 ns. Therefore, these specimens exhibit both intra and inter-tumor heterogeneity in the imaging features.

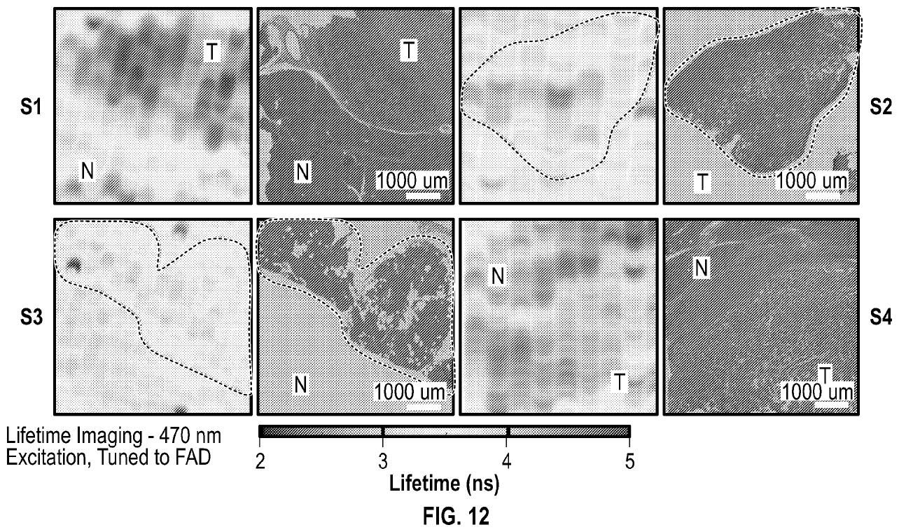

As with the MPM images, we also see relatively consistent features for the normal tissue. Normal pancreatic exocrine tissue has a lifetime of approximately 3.75 ns, whereas stromal tissues appear to have a higher lifetime of approximately 5 ns. FIG. 12 shows the 470 nm excitation channel, which has overall less variation in the lifetime, and may be used in combination with the 390 nm channel for calculating the optical redox ratio.

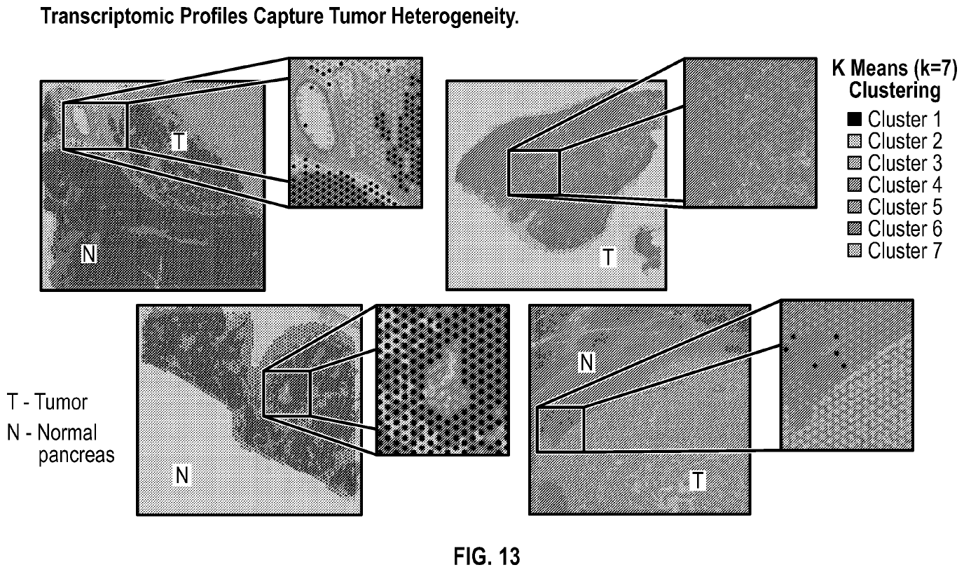

FIG. 13 shows the results of k-means clustering performed with k=7 for spatial transcriptome data. Some clear trends are visible: normal exocrine tissue consistently clusters in Cluster 1 among all three patients with normal tissue present, which agrees with the observation of both the MPM and FLIm features having consistent values for this tissue type. The stromal tissue clusters consistently into Cluster 3 for all specimens, once again consistent with the behavior of our imaging features. Interestingly, we see that there are separate subtypes identified through this clustering: all three patient specimens containing tumor cluster into different groups, and sample S2 (all tumor) exhibits two separate clusters within the single patient sample. Therefore, this approach clearly captures the heterogeneity that we observe in our imaging features, both between patients and within a single patient. These compelling results demonstrate that even with simple analytic techniques such as k-means clustering, we begin to identify heterogeneity in our patient specimens that is reflected in our label-free imaging markers.

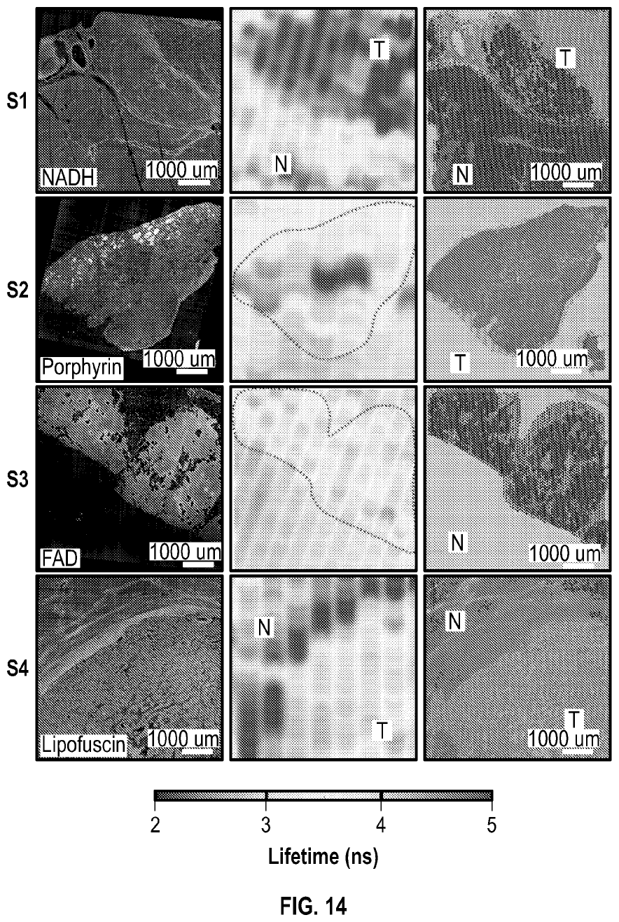

For a side-by-side comparison, FIG. 14 shows the MPM and FLIm images (390 nm channel) next to the respective clustered transcriptomic data. The transcriptomic clustering aligns with the qualitative variation that we observe in the label-free images. For example, sample S2 exhibits a higher lifetime in the center of the specimen, which corresponds to Cluster 5 of the transcriptomic data, whereas the rim corresponds to Cluster 4, with a much lower lifetime. Similarly, the texture observed in the MPM images appears significantly different qualitatively for all tumor specimens, and this is captured by the unique clustering that each sample exhibits. Therefore, this is a powerful approach to capture both within-patient and between-patient variability that may be lost when using standard validation approaches such as staining.

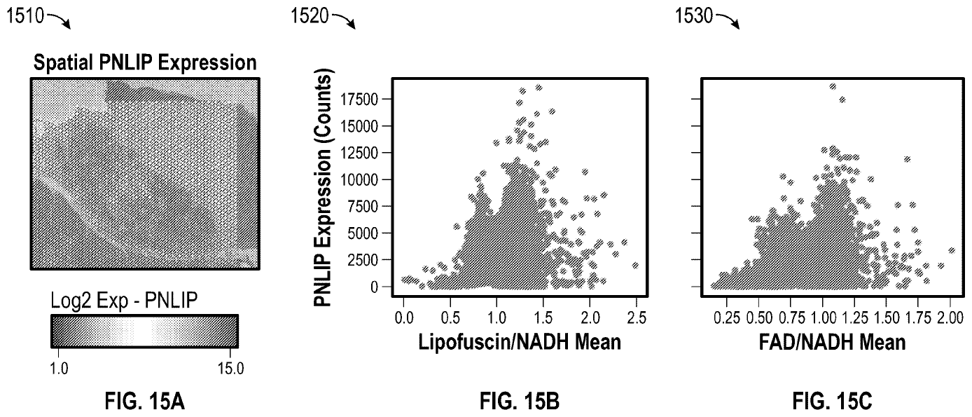

FIG. 15 includes plots 1510, 1520, and 1530. Plot 1510 shows an example of how visualizing a single spatial mapping of gene expression can be an equivalent, or even an improved, version of antibody staining to determine where certain makers are expressed. For example, the gene PNLIP encodes for a pancreatic lipase expressed by exocrine cells and is not expressed by tumor cells, as is clearly shown in FIG. 15. This simple scenario is nearly equivalent to staining the tissue with a fluorescent antibody for such a marker. It demonstrates that this approach may provide similar information to antibody staining-however, it is not exactly a one-to-one correlation due to possible post-translational modifications. However, in general, this approach offers a comprehensive library of genes and is not limited by multiplexing constraints. Another advantage of this approach is that the data readout is quantitative. Plots 1520 and 1530 show the result of a simple quantitative analysis performed for the pixel-to-pixel registration between transcriptomic and imaging modalities. By visualizing gene expression as a function of imaging marker value, we begin to see clusters that arise, illustrating that, for example, the tumor region corresponds to lower expression of PNLIP, and a lower average value of two of our imaging channels, whereas normal tissue has both higher imaging marker value and gene expression. The features plotted here are the average value for the ROI corresponding to each barcode point in the lipofuscin channel and FAD channel, each normalized by the average value of the NADH channel.

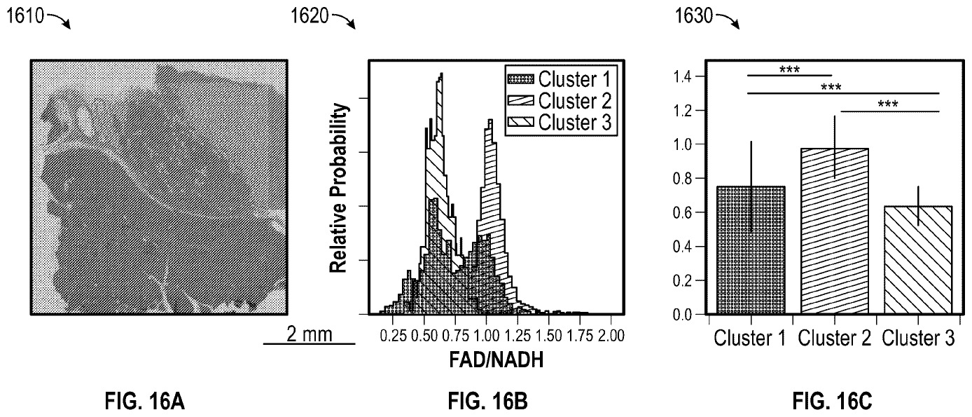

FIG. 16 illustrates how pixel-wise coregistration can enable more precise analysis of imaging markers for different tissue types, when compared to standard ROI selection guided by histopathology. FIG. 16 includes plots 1610, 1620, and 1630. Plot 1610 shows k-means clustering for patient S1 with k=3. Plot 1620 shows the average of the FAD channel normalized by the average of the NADH channel for each barcode point classified based on the k-means clustering and plotted as probability density distributions. Plot 1630 shows the average values of each distribution, with statistical significance denoted as *** for p<0.001. It is possible to infer that Cluster 1 is normal pancreatic tissue, Cluster 2 shows significant up regulation of transcripts associated with cancer signaling, including RAS oncogene family members, notably RAB3B, suggesting this spatial area is cancerous. Finally, Cluster 3 has a high expression of HBA2, which is associated with a hemoglobin protein alpha-globin, suggesting that this spatial area is dominated by blood cells.

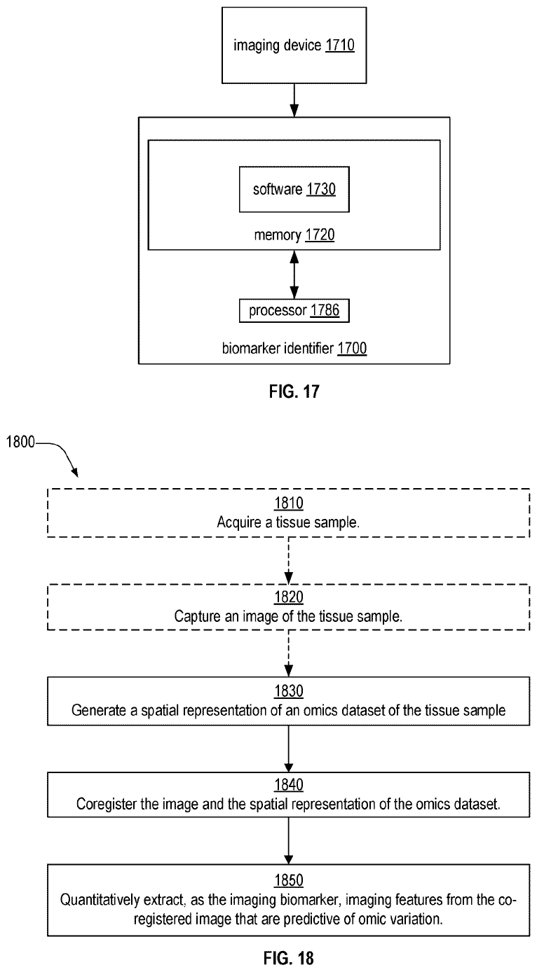

FIG. 17 is a functional block diagram of a biomarker identifier 1700. Biomarker identifier 1700 includes a processor 1786 and a memory 1720. Memory 1720 includes software 1730, which includes machine-readable instructions. Processor 1786 executes the machine-readable instructions to perform functions of biomarker identifier 1700 as described herein.

Memory 1720 may be transitory and/or non-transitory and may include one or both of volatile memory (e.g., SRAM, DRAM, computational RAM, other volatile memory, or any combination thereof) and non-volatile memory (e.g., FLASH, ROM, magnetic media, optical media, other non-volatile memory, or any combination thereof). Part or all of memory 1720 may be integrated into processor 1786.

FIG. 17 depicts an imaging device 1710 that is communicatively coupled to biomarker identifier 1700, e.g., via data acquisition hardware or communication interface hardware of biomarker identifier 1700. Biomarker identifier 1700 may also include imaging device 1710, which may be a microscope. Imaging device 1710 may execute one or more of the following imaging methods: autofluorescent imaging, fluorescence lifetime imaging, Raman imaging, hyperspectral imaging, polarization imaging, and medical contrast imaging.

FIG. 18 is a flowchart of a method 1800 for identifying an imaging biomarker of an image of a tissue sample. Method 1800 may be implemented within one or more aspects of biomarker identifier 1700. In embodiments, method 1800 is implemented by processor 1786 executing computer-readable instructions of software 1730. Method 1800 includes steps 1830, 1840, and 1850, and may also includes at least one of steps 1810 and 1830.

Step 1810 includes acquiring the tissue sample via one of laser capture microdissection, mechanical dissection, single-cell sequencing, and a combination thereof. Step 1820 includes capturing the image by one of autofluorescent imaging, fluorescence lifetime imaging, Raman imaging, hyperspectral imaging, polarization imaging, medical contrast imaging, and a combination thereof.

Step 1830 includes generating a spatial representation of an omics dataset of the tissue sample. In embodiments, the omics dataset includes one of genomic data, proteomic data, transcriptomic data, metabolomic data, and a combination thereof. In step 1830, generating a spatial representation may include spatially barcoding the tissue sample.

Step 1840 includes coregistering the image and the spatial representation of the omics dataset. The coregistering of step 1840 may include at least one of a pixel-wise coregistration, a cell-wise coregistration, and a bulk tissue coregistration.

Step 1850 includes quantitatively extracting, as the imaging biomarker, imaging features from the coregistered image that are predictive of omic variation. Step 1850 may employ one or more of the following feature extraction methods: Texture analysis (e.g., via grey-level co-occurrence matrix), intensity averaging, deep learning (e.g., convolutional neural networks), morphological descriptors (size and shape analysis), frequency analysis, and spectral signature extraction. Step 1850 may include determining a differentially expressed imaging feature between a first and a second clustered region of the plurality of clustered regions. The imaging biomarker may be used to predict omic expression in other tissue samples, or for diagnostic purposes.

Method 1800 may include steps of dimension reduction and clustering. Dimension reduction includes reducing a dimension of expression data, associated with the omics dataset, to yield reduced-dimension expression data. The expression data may be normalized before reducing its dimension. The Dimension reduction may utilize one or more of nonnegative matrix factorization and principal component analysis.

Clustering includes clustering, based on expression levels and not based on spatial information, barcode regions of the reduced-dimension expression data to yield a plurality of clustered regions. In such embodiments, step 1830 includes, for each of the plurality of clustered regions, generating a respective one of a plurality of two-dimensional representations of the clustered region using a nonlinear dimensionality-reduction technique.

The nonlinear dimensionality-reduction technique may be a stochastic neighbor embedding technique. The stochastic neighbor embedding technique may be a t-distributed stochastic neighbor embedding.

Changes may be made in the above methods and systems without departing from the scope of the present embodiments. It should thus be noted that the matter contained in the above description or shown in the accompanying drawings should be interpreted as illustrative and not in a limiting sense. Herein, and unless otherwise indicated, the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

We claim:

1. A method for identify an imaging biomarker of an image of a tissue sample, comprising:
generating a spatial representation of an omics dataset of the tissue sample;
coregistering the image and the spatial representation; and
quantitatively extracting, as the imaging biomarker, imaging features from the coregistered image that are predictive of omic variation.

2. The method of claim 1, further comprising:
reducing a dimension of expression data, associated with the omics dataset, to yield reduced-dimension expression data;
clustering, based on expression levels and not based on spatial information, barcode regions of the reduced-dimension expression data to yield a plurality of clustered regions; and
generating the spatial representation comprising, for each of the plurality of clustered regions, generating a respective one of a plurality of two-dimensional representations of the clustered region using a nonlinear dimensionality-reduction technique.

3. The method of claim 2, the nonlinear dimensionality-reduction technique being a stochastic neighbor embedding technique.

4. The method of claim 3, the stochastic neighbor embedding technique being a t-distributed stochastic neighbor embedding.

5. The method of claim 2, further comprising normalizing the expression data before reducing its dimension.

6. The method of claim 2, said reducing comprising reducing the dimension using nonnegative matrix factorization.

7. The method of claim 2, said reducing comprising reducing the dimension using principal component analysis.

8. The method of claim 2, quantitatively extracting comprising:
determining a differentially expressed imaging feature between a first and a second clustered region of the plurality of clustered regions.

9. The method of claim 1, further comprising:
capturing the image by one of autofluorescent imaging, fluorescence lifetime imaging, Raman imaging, hyperspectral imaging, polarization imaging, medical contrast imaging, and a combination thereof.

10. The method of claim 1, further comprising:
acquiring the tissue sample via one of laser capture microdissection, mechanical dissection, single-cell sequencing, spatial barcoding, and a combination thereof.

11. The method of claim 1, the omics dataset including one of genomic data, proteomic data, transcriptomic data, metabolomic data, and a combination thereof.

12. The method of claim 1, said coregistering being one of a pixel-wise coregistration, a cell-wise coregistration, and a bulk tissue coregistration.

13. The method of claim 1, generating a spatial representation comprising spatially barcoding the tissue sample.

14. A biomarker identifier comprising:
a processor; and
a memory storing machine-readable instructions that, when executed by the processor, cause the processor to identify an imaging biomarker of an image of a tissue sample by:
generating a spatial representation of an omics dataset of the tissue sample;
coregistering the image and the spatial representation; and
quantitatively extracting, as the imaging biomarker, imaging features from the coregistered image that are predictive of omic variation.

15. The biomarker identifier of claim 14, the memory further storing machine-readable instructions that, when executed by the processor, cause the processor to:
reduce a dimension of expression data, associated with the omics dataset, to yield reduced-dimension expression data;
cluster, based on expression levels and not based on spatial information, barcode regions of the reduced-dimension expression data to yield a plurality of clustered regions; and
generate the spatial representation comprising, for each of the plurality of clustered regions, generating a respective one of a plurality of two-dimensional representations of the clustered region using a nonlinear dimensionality-reduction technique.

16. The biomarker identifier of claim 15, the memory further storing machine-readable instructions that, when executed by the processor, cause the processor to, before reducing the dimension, normalizing the expression data.

17. The biomarker identifier of claim 15, the memory further storing machine-readable instructions that, when executed by the processor, cause the processor to, when reducing the dimension, reduce the dimension using nonnegative matrix factorization.

18. The biomarker identifier of claim 15, the memory further storing machine-readable instructions that, when executed by the processor, cause the processor to, when reducing the dimension, reduce the dimension using principal component analysis.

19. The biomarker identifier of claim 15, the memory further storing machine-readable instructions that, when executed by the processor, cause the processor to, when quantitatively extracting, determining a differentially expressed imaging feature between a first and a second clustered region of the plurality of clustered regions.

20. The biomarker identifier of claim 14, the memory further storing machine-readable instructions that, when executed by the processor, cause the processor to, when coregistering, coregister the image and the spatial representation via one of a pixel-wise coregistration, a cell-wise coregistration, and a bulk tissue coregistration.
